(19) United States
(12) Patent Application Publication
(10) Pub. No.: US 2009/0170808 A1
(43) Pub. Date: Jul. 2, 2009

(54) ORAL AGENT FOR IMPROVING AND PROTECTING THE FUNCTION OF JOINT COMPRISING HYALURONIC ACID-PHOSPHOLIPID COMPLEXES

(76) Inventors: Peixue Ling, Shandong (CN); Hiafeng Yin, Shandong (CN); Qiuyan Jiang, Shandong (CN); Tianmin Zhang, Shandong (CN)

Correspondence Address: FISH & RICHARDSON P.C., PO BOX 1022, MINNEAPOLIS, MN, 55440-1022, US

(21) Appl. No.: 12/091,918

(22) PCT Filed: Oct. 27, 2006
(86) PCT No.: PCT/CN06/02884

§ 371 Date: Aug. 19, 2008

(30) Foreign Application Priority Data
Oct. 28, 2005 (CN) 200510118946.9

(51) Int. Cl. A61K 31/728; C07H 1/00

(52) U.S. Cl. 514/54; 536/123.1

(57) ABSTRACT

The present invention relates to an oral formulation comprising a hyaluronic acid-phospholipid complex, and to use of a hyaluronic acid-phospholipid complex for the manufacture of a joint function-improving and protecting agent for oral administration which can alleviate the arthritic symptoms in patients with arthritic conditions, increase the concentration of hyaluronic acid in synovial fluid, improve the lubrication of joints, as well as maintain and enhance the normal functions of joints.

### TECHNICAL FIELD

The present invention relates to an oral formulation comprising a hyaluronic acid-phospholipid complex, and to use of a hyaluronic acid-phospholipid complex for the manufacture of a joint function-improving and protecting agent which can alleviate the arthritic symptoms in patients with arthritic conditions, increase the concentration of hyaluronic acid in synovial fluid, improve the lubrication of joints, as well as maintain and enhance the normal functions of joints via oral administration.

### BACKGROUND ART

Osteoarthritis (OA) is a clinically common joint disease. OA mostly occurs in aging people, and thus is known as “disease in the latter years of life”. With the increasing average life-span, the prevalence of OA in aging people continues to rise. It seriously impairs working ability and becomes the second leading disabling disease, only next to heart disease, in people above 50 years of age.

Hyaluronic acid (HA), which is an endogenous macromolecular mucopolysaccharide, is found mainly in connective tissues such as skin, cartilage, synovial fluid and cornea, and has an average relative molecular weight (Mr) of 1×10⁵−10⁷. Hyaluronic acid has significant efficacy in the treatment of ocular diseases and joint diseases due to its unique lubricating property and viscoelastic property. In addition, hyaluronic acid acts in vivo to hold water, adjust osmotic pressure, promote wound healing and scavenge oxygen free radicals.

At present, hyaluronic acid has been widely used in medical fields such as opthalmology, orthopedics and dermatology as well as in health care food and cosmetics. However, hyaluronic acid is used in the treatment of arthritic conditions mainly via injection into joint cavity. For example, sodium hyaluronate (SH) injection has already been used in bone and joint diseases as a viscoelasticity-supplementing therapeutic agen. Said injection is directly injected into the diseased joint to treat OA, restoring rheology of synovial fluid and joint tissue matrix, stabilizing internal environment, increasing joint lubrication, alleviating synovitis and enhancing self secretion of hyaluronic acid, to thereby reduce damage to articular cartilage, alleviate clinical symptoms and improve function. SH exhibits good therapeutic effect in mild and moderate OA, but is much less effective in serious and advanced OA. In addition, the intra-articular injection of hyaluronic acid is to be performed under sterile conditions by a skilled physician. Said procedure is painful, leading to poor compliance. On the other hand, such substances are poorly absorbed from gastrointestinal tract upon oral administration, mainly because of high relative molecular weight, poor liposolubility, difficulty in crossing biomembrane barrier, the presence of polysaccharide-degrading enzymes in the gastrointestinal tract, and the like.

Thus, there is still a need in the art for a joint function-improving and protecting agent which is more convenient to administer and may be better adsorbed.

The present invention solves the above technical problem by providing a hyaluronic acid-phospholipid complex that can be orally administered. Upon complexing, the phospholipid increases the lipophilicity of hyaluronic acid. Also, by taking advantage of the high affinity of phospholipids toward cell membrane, the binding of hyaluronic acid molecule to cell membrane and thus the absorption of hyaluronic acid is enhanced, leading to prolonged duration of action, and improved oral bioavailability of hyaluronic acid. Upon digestion and absorption of hyaluronic acid, the synthetic precursors of hyaluronic acid are increased, and the hyaluronic acid deficiency in internal organs and tissues is supplemented, to achieve systemic action, in particular to increase the concentration of hyaluronic acid in joints of a patient with arthritic conditions, to increase the viscosity of synovial fluid, and to alleviate the symptoms of arthritic conditions. Based on the above theory, a hyaluronic acid-phospholipid complex is used according to the present invention to be orally administered as a joint function-improving and protecting agent.

### Contents of the Invention

The present invention provides an oral joint function-improving and protecting agent comprising a hyaluronic acid-phospholipid complex. The complexed hyaluronic acid and phospholipid possess both the properties of phospholipids and those of hyaluronic acid, and the phospholipid moiety facilitates the absorption and achieves a slow-release of hyaluronic acid. Said complex can reduce joint inflammation, increase the concentration of hyaluronic acid in the synovial fluid, and increase the viscosity of synovial fluid, to thereby improve joint function in patients with arthritic conditions.

As used herein, the term “improving and protecting joint function” or “joint function-improving and protecting” mainly refers to the effects of alleviating a range of joint symptoms such as pain, swelling and morning stiffness, enhancing joint mobility and reducing inflammation of the synovial membrane; stimulating chondrocytes to produce normal long-chain structured proteoglycans, as well as inhibiting other articular cartilage-damaging substances, to thereby promote the anabolic metabolism of chondrocytes, and repair the aging or damaged cartilage matrix. As a result, the normal morphology and function of chondrocytes may be restored, the articular cartilages may regain their smoothness and elasticity, thus slowing the aging of articular cartilages, controlling disease progression, and eliminating the symptoms of osteoarthritis.

The arthritic conditions that can be treated according to the present invention include osteoarthritis, rheumatic arthritis and other various joint dysfunctions. Also, the joint function-improving and protecting agent according to the present invention can be administered orally for joint health care, by slowing joint function degeneration due to increasing age, improving joint lubrication, maintaining normal function of joints, and preventing degenerative changes in joints.

Therefore, in one aspect, the present invention provides an oral formulation comprising a hyaluronic acid-phospholipid complex as the active ingredient.

In another aspect, the present invention provides use of a hyaluronic acid-phospholipid complex in the manufacture of a product for improving and protecting joint function.

The hyaluronic acid-phospholipid complex useful in the present invention may be prepared by thoroughly mixing hyaluronic acid or carbodiimide-activated hyaluronic acid in the form of powder or aqueous solution with an aqueous dispersion of phospholipids, and stirring at a constant temperature to obtain the complex of the present invention, wherein the aqueous dispersion of phospholipid may be obtained by a process for preparing liposomes, or by direct hydration of phospholipid through mechanical stirring, vortexing and ultrasonication.

In the process for preparing the hyaluronic acid-phospholipid complex according to the present invention, the safe proportion of hyaluronic acid to phospholipid is 1:0.1 to 1:10, the reaction temperature is 28-45° C., preferably 30-40° C., and the reaction time is 2-48 hrs, preferably 4-12 hrs.

In the process for preparing the hyaluronic acid-phospholipid complex according to the present invention, the solvent for hyaluronic acid and the aqueous dispersion medium for phospholipid may be physiological saline or phosphate buffer.

In one embodiment, the process for preparing the hyaluronic acid-phospholipid complex comprises the steps of dissolving hyaluronic acid and phospholipid, forming a film of phospholipid, hydrating the phospholipid film, mixing hyaluronic acid and the phospholipid dispersion, and stirring the mixed solution at a constant temperature to effectuate complexation, to thereby obtain the complex of the present invention.

In one embodiment, the phospholipid dispersion may be prepared by subjecting an organic solution of phospholipid to rotary evaporation to form a film before adding a dispersion medium, and hydrating the film by one or more methods selected from the group consisting of mechanical stirring, vortexing and ultrasonication, to obtain a homogeneous dispersion system.

In another embodiment, the phospholipid may first be formulated into liposomes by conventional methods, to which hyaluronic acid is then added, followed by stirring at a constant temperature to effectuate complexation, to thereby obtain the complex of the present invention.

In one embodiment, hyaluronic acid may be directly added both as an aqueous solution and as powder to the dispersion of phospholipid.

In another embodiment, hyaluronic acid may be activated with carbodiimide before adding to the dispersion of phospholipid.

The hyaluronic acid useful in the present invention is a water soluble polyanionic mucopolysaccharide, and can form certain interchain or intrachain hydrophobic regions and hydrophilic regions upon dissolution in an aqueous solution. The phospholipid useful in the present invention is an amphipathic substance, having two hydrophobic long fatty acid chains and one hydrophilic phosphate chain. The formation of the hyaluronic acid-phospholipid complex according to the present invention has been verified by IR spectrum and differential scanning calorimetry (DSC) curves. Hyaluronic acid and phospholipid can bind to form a complex (not a simple mixture thereof) through hydrophobic binding, electrostatic bonding, hydrogen bonding and the like. As determined, in the hyaluronic acid-phospholipid complex according to the present invention, the mass ratio of phospholipid to hyaluronic acid being complexed is 0.08 to 0.5.

The hyaluronic acid to be used according to the present invention may be derived from animal tissue extract, microbial fermentation and genetic engineering, and includes hyaluronic acid and physiologically acceptable salts thereof, including, but not limited to, sodium, potassium, calcium and zinc salts.

In addition, the hyaluronic acid to be used according to the present invention has a relative molecular mass of from 1×10⁴ to 3×10⁶, preferably <1×10⁶.

The phospholipid to be used according to the present invention is any single one phospholipid or a mixture of more than one phospholipids selected from the group consisting of: phosphatidates; phosphatidyichlolines (lecithin); phosphatidylethanolamines (cephalin); phosphatidylserines; N-methylethanolamine glycerol phosphates; N, N-dimethylethanolamine glycerol phosphates; N-acylethanolamine glycerol phosphates; N-2(hydroxyethyl)alanine phosphatidyl glycerides; biphosphatidyl glycerides; glycerol phosphate; glucosamine glycerol phosphate glyceride; O-amino acid phospholipid glyceride; phosphatidylinositols; phosphatidylinositol phosphate; phosphatidylinositol diphosphate; phosphoinositol triphosphate; phosphatidylglucose; diglucose glycerol phosphatidate; sphingosylglycolipid; glycerylglycolipid; cerebroside; ganglioside; monoglycosyl cerebroside; sphingomyelin; synthetic phospholipids, such as dipalmitoyl phosphatidylcholine, dipalmitoyl phosphatidylethanolamine; lysophospholipids; and the like, preferably phosphatidates, phosphatidylchlolines (lecithin), phosphatidylethanolamines (cephalin), phosphatidylserines, glycerol phosphate, phosphatidylinositols, sphingomyelin, dipalmitoyl phosphatidylcholine, dipalmitoyl phosphatidylethanolamine, and lysophospholipids, more preferably phosphatidates, phosphatidylchlolines, phosphatidylethanolamines, dipalmitoyl phosphatidylcholine, dipalmitoyl phosphatidylethanolamine, and lysophospholipids, and most preferably phosphatidylchlolines, phosphatidylethanolamines, and lysophospholipids.

The oral formulation comprising a hyaluronic acid-phospholipid complex according to the present invention may be in various forms well known to a person skilled in the art, including oral solid formulations, such as tablets, capsules, pills, pellicles, granules, powders and the like; oral liquid formulations, such as oral solutions, suspensions, emulsions, gels, pastes and the like.

In the oral formulation according to the present invention, the hyaluronic acid-phospholipid complex is present in an amount of from 0.01 to 0.5 g per 1 g or 1 ml of the formulation. Further nutritional supplements or other active ingredients, such as chondroitin sulfate and aminoglucoses, may be optionally added as required, in addition to the basic composition of the oral formulation according to the present invention. Aminoglucoses include aminoglucose hydrochloride, aminoglucose sulfate, and the like.

The oral formulation according to the present invention may be orally administered as such, or may be dispersed in physiological saline, phosphate buffer solution or carbonate buffer solution, or even added in foods.

### DESCRIPTION OF THE FIGURES

FIG. 1 shows the infrared spectra of hyaluronic acid, a phospholipid, a mixture of hyaluronic acid and phospholipid, and a hyaluronic acid-phospholipid complex of the present invention. A: hyaluronic acid; B: lecithin; C: a physically ground mixture (hyaluronic acid: lecithin=1:3); D: the complex of Preparation Example 1 according to the present invention.

FIG. 2 shows the DSC curves of hyaluronic acid (HA), lecithin (PL), a mixture of hyaluronic acid and phospholipid, and a hyaluronic acid-phospholipid complex according to the present invention.

### MODE OF CARRYING OUT THE INVENTION

The following examples are now provided to further illustrate the present invention, but are not to be interpreted in any way as limiting the scope of the invention.

### Examples

### Preparation Example 1

### Preparation and Identification of Hyaluronic Acid-Phospholipid Complex 1

1.2 g sodium hyaluronate was accurately weighed and added slowly to 100 ml phosphate buffer with stirring until complete dissolution. 3.6 g lecithin (Lipoid E 80 from Lipoid Co., Germany, which contains about 80% phosphatidylchloline, about 8% phosphatidylethanolamine, and small amounts of sphingomyelin, lysophospholipid and the like) was accurately weighed and dissolved in anhydrous ethanol. The ethanol was then removed by rotary evaporation, leaving a film of phospholipid on the flask wall, followed by vacuum drying to completely remove ethanol. The resulting phospholipids film was hydrated by addition of 100 ml phosphate buffer with mechanical stirring, and was ultrasonicated for 10 min to obtain a homogenous dispersion, before adding to the previously prepared hyaluronic acid solution. 6 hrs of mechanical stirring at a constant temperature of 37° C. yielded the hyaluronic acid-phospholipid Complex 1. In this example, the mass ratio of the starting hyaluronic acid to phospholipid is 1:3.

The determination of the complexation state of phospholipid and hyaluronic acid in the complex:

An emulsion of the complex was prepared and then freeze dried under vacuum. Before measurement, the freeze dried complex was accurately weighed (m₁; the amounts of hyaluronic acid and lecithin therein, marked as m_{HA} and m_{PL} respectively, were calculated according to their proportion). Then a certain amount of chloroform based on the proportion of lecithin in the complex was added, followed by shaking for 10 min, suction filtering under decreased pressure, and drying of the chloroform-insoluble material, which was then accurately weighed (m₂).

The mass ratio of phospholipid to hyaluronic acid being complexed=[m_{PL}−(m₁−m₂)]/m_{HA}.

In the present example, the mass ratio of phospholipid to hyaluronic acid being complexed was 0.4733.

The formation of Complex 1 was confirmed by using attenuated total reflectance IR spectrum (MB-HATR) and differential scanning calorimetry (DSC), respectively.

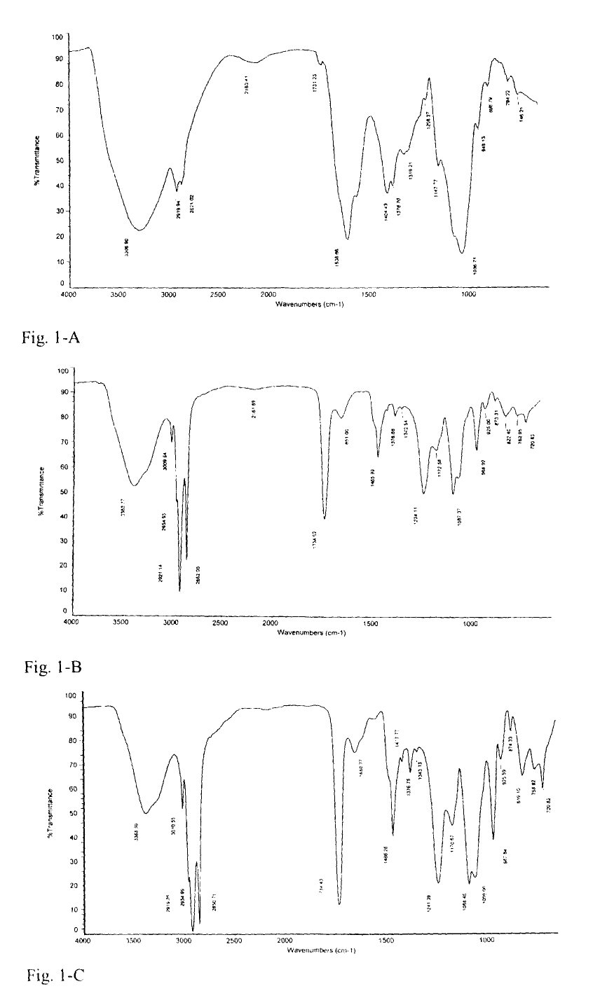

MB-HATR: The sample was spread on the KBr pellet and pressed tight. The spectral resolution was 8.0 cm. The average value of 200 runs was taken, and the scanning range was from 4000 to 500 cm⁻¹. The results were shown in FIG. 1.

It can be seen from FIG. 1, there are significant changes in the relative strength of some adjacent peaks for the complex, as compared with the mixture. The absorption peaks of C=O and C—N (amido group) of hyaluronic acid in the complex shifted to higher wave number, and the IR absorption of dissociated carboxyl group (ν_{O—C═O}) of hyaluronic acid has also somewhat changed. The stretching vibration peaks (ν_{P═O} and ν_{P═O—C}) of P—O bonds at polar ends of phospholipid in the complex shifted (ν_{P═O} shifted to higher wave number, and ν_{P—O—C} shifted to lower wave number). The band position of IR absorption of amido group (ν_{C═O} and ν_{C—N}) and dissociated carboxyl group of hyaluronic acid has changed, suggesting that the interaction between them might occur between the carboxyl or amido group of hyaluronic acid and the polar end of phospholipid, and belongs to ionic bonding and hydrophobic action. In addition, the absorption band of hydroxyl in the complex was broader than in the mixture, and the wave number of the absorption band is lower than the mixture, indicating that new hydrogen bonds might have formed in the complex.

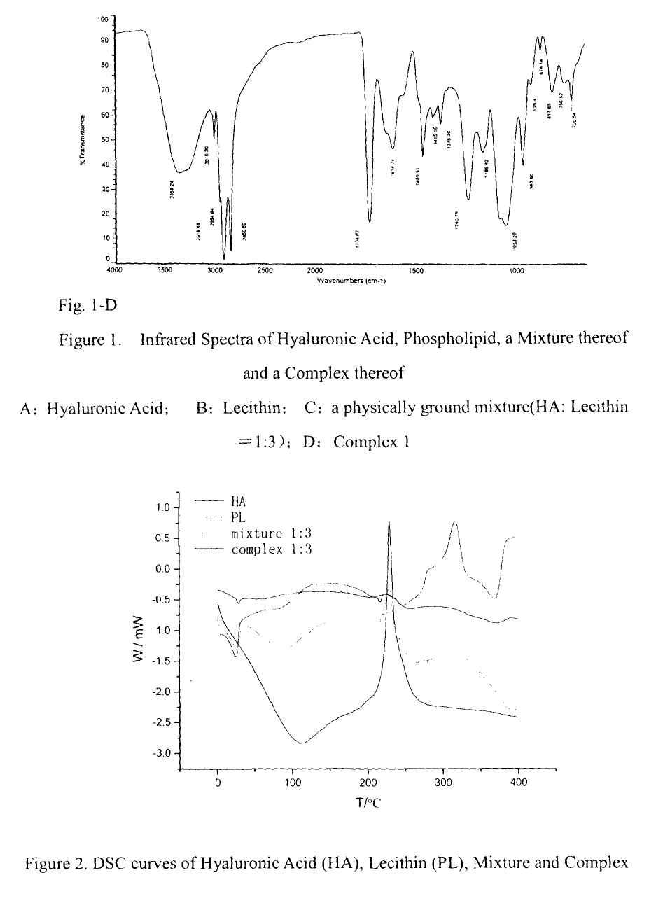

The operation conditions for DSC were: a N₂ flow of 50 ml/min, a temperature range of 0-400° C., and a heating rate of 5° C./min. The results were shown in FIG. 2.

It can be seen from FIG. 2 that in the DSC curve for the complex, the endothermic peak at about 26° C. disappeared, indicating that, after forming complex, phospholipid no longer undergoes phase change due to the interaction between hyaluronic acid and phospholipid; and the strong exothermic peak at 317.79° C. also disappeared, possibly because hyaluronic acid, as a polysaccharide, is a good protector for stabilizing liposome film, and thus can protect phospholipid against oxidative decomposition under high temperatures. In contrast, the DSC curve of the mixture was basically a superposition of the DSC curves of hyaluronic acid and phospholipid.

Therefore, the above results showed that hyaluronic acid and phospholipid formed a complex.

### Preparation Example 2

### Preparation and Identification of Hyaluronic Acid-Phospholipid Complex 2

The procedures for preparing Complex 2 were the same as those for preparing Complex 1, except that 1.2 g sodium hyaluronate and 0.24 g phospholipid used in Example 1 respectively were accurately weighed. In this example, the mass ratio of the starting hyaluronic acid to phospholipid is 1:0.2. The complexation state of phospholipid and hyaluronic acid in Complex 2 was determined as described for Complex 1, and the result showed that the mass ratio of phospholipid to hyaluronic acid being complexed was 0.1886. Complex 2 was identified by the same methods as those used for Complex 1, and the results showed that its IR absorption profile and change in thermochemical properties were substantially the same as Complex 1.

### Preparation Example 3

### Preparation and Identification of Hyaluronic Acid-Phospholipid Complex 3

The procedures for preparing Complex 3 were the same as those for preparing Complex 1, except that 1.2 g sodium hyaluronate and 10.8 g phospholipid used in Example 1 respectively were accurately weighed. In this example, the mass ratio of the starting hyaluronic acid to phospholipid is 1:9. The complexation state of phospholipid and hyaluronic acid in Complex 3 was determined as described for Complex 1, and the result showed that the mass ratio of phospholipid to hyaluronic acid being complexed was 0.4805. Complex 3 was identified by the same methods as those used for Complex 1, and the results showed that its IR absorption profile and change in thermochemical properties were also substantially the same as Complex 1.

### Formulation Example 1

The complex obtained in Preparation Example 1 was formulated into a tablet according to the formula shown in Table 1.

TABLE 1
Formula of tablets

| Components | wt % |
|---|---|
| Hyaluronic acid-phospholipid complex | 60.0 |
| Chondroitin sulfate | 16.0 |
| Starch | 12.0 |
| Starch slurry | 8.0 |
| Magnesium stearate | 4.0 |

The tablet was prepared by the steps of deaggregating the hyaluronic acid-phospholipid complex and chondroitin sulfate and an amount of starch; mixing with starch slurry, followed by granulating; mixing the obtained dry granules with magnesium stearate and the remaining starch, and then subjecting the resulting mixture to tabletting in a tablet press.

### Formulation Example 2

The complex obtained in Preparation Example 2 was formulated according to the composition as shown in Table 2 into granules of a certain particle size following conventional processes, which are then packaged into pouches.

TABLE 2
Formula of granules

| Components | wt % |
|---|---|
| Hyaluronic acid-phospholipid complex | 69.0 |
| Lactose | 21.0 |
| Microcrystalline cellulose | 10.0 |

### Formulation Example 3

The complex obtained in Preparation Example 3 was formulated according to the composition as shown in Table 3 into an oral solution using conventional processes.

TABLE 3
Formula of an oral solution

| Components | Weight (g) |
|---|---|
| Hyaluronic acid-phospholipid complex | 0.5 |
| Gelatin | 1.0 |
| Vitamin Mix | 1.2 |
| Tween | 2.0 |
| Syrup | 2.5 |
| Potassium sorbate | 0.05 |
| Distilled water | q.s. to 100 ml |

### Animal Experiment

### 1. Methods

30 rabbits were randomly divided into five groups, i.e., normal control group; normal saline (NS) injection group; hyaluronic acid (HA) oral administration group; hyaluronic acid (HA) injection group; and hyaluronic acid-phospholipid complex (HA-PL, the complex of Preparation Example 2) oral administration group.

Using the papain-induced OA model in rabbit, the latter four groups of rabbits received injection of 0.1 ml of a papain solution (1.8 mg papain and 50 mg cysteine hydrochloride in 1 ml normal saline, sterile filtered through a 0.22 μm filter membrane) into the right knee articular cavity. A second injection was performed 3 days later.

Starting on Day 7 post-modeling, the rabbits were dosed. For administration by injection into joint cavity, a injection volume of 0.3 ml, and a hyaluronic acid concentration of 10 mg/ml were used, once every five days for a total of 3 injections during the test. For oral administration, the respective agent was given intragastrically every day, at a dosage 40 mg/kg body weight, calculated as hyaluronic acid, in a volume of 1 ml/100 g body weight, for 2 weeks.

After completion of the administration, synovial fluid was aspirated, and the rabbits were sacrificed. The knee of the rabbit was shaved, and the joint cavity was opened for visual inspection as a whole. Then the cartilage was removed in its entirety. Glycosaminoglycan (GAG) was extracted by hydrolysis using diluted alkali and protease hydrolysis and ultracentrifugation. The total amount of GAG in the cartilage was determined by the azure A method, and the level of HA in the synovial fluid was determined using an HA RIA kit.

The content of GAG in samples from each group was expressed as the amount contained in every 1 mg wet weight of the cartilage ( x̅±s), and was analyzed by t-test for significance test.

2. The GAG Content in Samples from Each Group

The results of determination were reported in Table 4. The GAG content in the NS group decreased significantly, suggesting that proteoglycan (PG), the main component thereof being GAG, was lost from the matrix in OA. The difference was significant (P<0.05) as compared with the other treatment groups. The HA-PL oral administration group showed a significant difference (P<0.05) as compared with the HA oral administration group, and was comparable to the HA injection group. The above results demonstrated that the hyaluronic acid-phospholipid complex could reduce the degenerative changes in cartilages, and effectively inhibit the decrease of GAG content in cartilages. With the therapeutic effect being comparable to that in the HA injection group, the compliance of the patients to oral administration will obviously be better than to injection.

TABLE 4
GAG Content in knee joint cartilages
from different animal groups.

| Animal Grouping | GAG Content/(ng · ml⁻¹) |
|---|---|
| Normal | 32.9 ± 3.2¹⁾ |
| NS | 18.3 ± 3.7 |
| HA, oral administration | 23.3 ± 4.2¹⁾ |
| HA, injection | 29.9 ± 4.4^{1),2)} |
| HA-PL, oral administration | 31.3 ± 5.1^{1),2)} |

n = 6, x̅ ± s
Notes:
¹⁾P < 0.05 vs. the NS group;
²⁾P < 0.05 vs. the HA oral administration group

3. The HA Content in Knee Joint Synovial Fluid from Each Group of Animals

The results of determination were reported in Table 5. The HA content in the NS group decreased significantly, and the difference was significant (P<0.05) as compared with the other groups. The HA content in each treatment group was not significantly different from that in the normal group, indicating that the use of HA alone and of the hyaluronic acid-phospholipid complex could both increase the HA level in knee joint synovial fluid in rabbit OA model.

TABLE 5
HA Content in knee joint synovial fluid
from different animal groups.

| Animal Grouping | HA content/(mg · ml⁻¹) |
|---|---|
| Normal | 4.33 ± 0.60¹⁾ |
| NS | 3.39 ± 0.43 |
| HA, oral administration | 4.65 ± 0.54¹⁾ |
| HA, injection | 4.64 ± 0.51¹⁾ |
| HA-PL, oral administration | 4.70 ± 0.28¹⁾ |

n = 6, x̅ ± s
Notes:
¹⁾P < 0.05 vs. the NS group

### Clinical Efficacy of the Oral Formulation According to the Present Invention in Patients

19 OA patients aged 52±16 were investigated using the product of Formulation Example 2, with placebo as control, wherein the placebo group consisted of 9 patients and the treatment group consisted of 10 patients. The dosage was 10 mg/kg body weight/day, calculated as hyaluronic acid, twice a day given orally. After one month, the alleviation of joint symptoms and the improvement of joint function were evaluated, and the results were reported in Table 6.

TABLE 6
Joint conditions in different groups

| Indices | Treatment group | Placebo group |
|---|---|---|
| alleviation in anchylosis | 7 | 1 |
| alleviation in arthralgia | 8 | 2 |
| adverse gastrointestinal | 0 | 1 |
| effect upon oral | | |
| administration | | |
| overall improvement in | 9 | 2 |
| joint conditions | | |
| overall deterioration in | 0 | 1 |
| joint conditions | | |
| no change in joint | 1 | 6 |
| conditions | | |

1. Use of a hyaluronic acid-phospholipid complex in the manufacture of a product for improving and protecting joint function.

2. The use according to claim 1, wherein the product is an oral formulation.

3. The use according to claim 1 or 2, wherein the mass ratio of phospholipid to hyaluronic acid being complexed is 0.08 to 0.5 in the hyaluronic acid-phospholipid complex.

4. The use according to claim 1 or 2, wherein the hyaluronic acid is hyaluronic acid or a physiologically acceptable salt thereof, and has a relative molecular mass of from 1×10⁴ to 3×10⁶, preferably <1×10⁶; and the phospholipid is one or more selected from the group consisting of phosphatidates; phosphatidylchlolines; phosphatidylethanolamines; phosphatidylserines; N-methylethanolamine glycerol phosphates; N,N-dimethylethanolamine glycerol phosphates; N-acylethanolamine glycerol phosphates; N-2(hydroxyethyl)alanine phosphatidyl glycerides; biphosphatidyl glycerides; glycerol phosphate; glucosamine glycerol phosphate glyceride; O-amino acid phospholipid glyceride; phosphatidylinositols; phosphatidylinositol phosphate; phosphatidylinositol diphosphate; phosphoinositol triphosphate; phosphatidylglucose; diglucose glycerol phosphatidate; sphingosylglycolipid; glycerylglycolipid; cerebroside; ganglioside; monoglycosyl cerebroside; sphingomyelin; synthetic phospholipids including dipalmitoyl phosphatidylcholine, dipalmitoyl phosphatidylethanolamine; and lysophospholipids.

5. An oral formulation comprising a hyaluronic acid-phospholipid complex as the active ingredient.

6. The oral formulation according to claim 5, wherein the mass ratio of phospholipid to hyaluronic acid being complexed is 0.08 to 0.5 in the hyaluronic acid-phospholipid complex.

7. The oral formulation according to claim 5, wherein the hyaluronic acid is hyaluronic acid or a physiologically acceptable salt thereof, and has a relative molecular mass of from 1×10⁴ to 3×10⁶, preferably <1×10⁶; and the phospholipid is one or more selected from the group consisting of phosphatidates; phosphatidylchlolines; phosphatidylethanolamines; phosphatidylserines; N-methylethanotamine glycerol phosphates; N,N-dimethylethanolamine glycerol phosphates; N-acylethanolamine glycerol phosphates; N-2(hydroxyethyl)alanine phosphatidyl glycerides; biphosphatidyl glycerides; glycerol phosphate; glucosamine glycerol phosphate glyceride; O-amino acid phospholipid glyceride; phosphatidylinositols; phosphatidylinositol phosphate; phosphatidylinositol diphosphate; phosphoinositol triphosphate; phosphatidylglucose; diglucose glycerol phosphatidate; sphingosylglycolipid; glycerylglycolipid; cerebroside; ganglioside; monoglycosyl cerebroside; sphingomyelin; synthetic phospholipids including dipalmitoyl phosphatidylcholine, dipalmitoyl phosphatidylethanolamine; and lysophospholipids.

8. The oral formulation according to claim 5, further comprising nutritional supplements or other active ingredients, such as chondroitin sulfate and aminoglucose.

9. The oral formulation according to claim 8, which is an oral solid formulation, such as tablet, capsule, pill, pellicle, granules, and powders; or an oral liquid formulation, such as oral solution, suspension, emulsion, gel, and paste.

10. The oral formulation according to claim 5, comprising 0.01 to 0.5 g of the hyaluronic acid-phospholipid complex per 1 g or 1 ml of the formulation.
